(19) United States
(12) Patent Application Publication
(10) Pub. No.: US 2023/0105923 A1
(43) Pub. Date: Apr. 6, 2023

(54) A METHOD AND SYSTEM FOR INTRODUCING ONE OR MORE EXOGENOUS SUBSTANCES INTO AN IMMUNE CELL

(71) Applicant: AGENCY FOR SCIENCE, TECHNOLOGY AND RESEARCH, Singapore (SG)

(72) Inventors: Weijie Cyrus BEH, Singapore (SG); Yiqi SEOW, Singapore (SG)

(21) Appl. No.: 17/914,408

(22) PCT Filed: Mar. 26, 2021
(86) PCT No.: PCT/SG2021/050171

§ 371 Date: Sep. 26, 2022

(30) Foreign Application Priority Data
Mar. 26, 2020 (SG) 10202002812Q

(51) Int. Cl. C12N 15/87; C12N 5/0783; C12M 1/42; C12M 1/12

(52) U.S. Cl. CPC C12N 15/87; C12M 25/02; C12M 35/04; C12N 5/0636; C12N 2501/2302

(57) ABSTRACT

The present invention relates to a method and system for introducing one or more exogenous substances into an immune cell. The method comprises the steps of: providing a membrane having a plurality of pores configured to allow the immune cell to pass through, wherein the plurality of pores has an average pore diameter which is no less than an average diameter of the immune cell nucleus and no more than an average diameter of the immune cell, passing a fluid containing the immune cell and the on e or more exogenous substances through the plurality of pores to induce a mechanical stress to the immune cell and facilitate introduction of the one or more exogenous substances into the immune cell, and recirculating the fluid containing the immune cell and the one or more exogenous substances such that the same immune cell passes through the membrane more than once. In some embodiments, the immune cell is T-cells and the exogenous substance is mRNA.

### TECHNICAL FIELD

The present disclosure relates broadly to a method and a system for introducing one or more exogenous substances into an immune cell.

### BACKGROUND

Delivery of macromolecules such as polysaccharides, proteins, or nucleic acids into the cytoplasm of a cell can temporarily or permanently alter cell function for research or therapeutic purposes. However, existing techniques for intracellular delivery are limited, particularly for immune cells such as lymphocytes.

Currently known techniques for intracellular delivery include electroporation, viral vectors, transmembrane peptides, liposomes and polymersomes. However, electroporation results in considerable cytotoxicity. Viral vectors cannot infect resting lymphocytes. Transmembrane peptides do not efficiently transfect primary lymphocytes. Liposomes and polymersomes are workhorses for the production process of cell therapies, but residual amount of liposomal and polymeric material can affect cell function in unknown ways in vivo. Many of these techniques also result in the accumulation of non-biodegradable packaging or delivery materials within the cell that can affect cell function.

Thus, there is a need for a method and a system for introducing one or more exogenous substances into an immune cell that seek to address or at least ameliorate one or more of the above problems.

### SUMMARY

In one aspect, there is provided a method of introducing one or more exogenous substances into an immune cell, the method comprising, providing a membrane having a plurality of pores configured to allow the immune cell to pass through, wherein the plurality of pores has an average pore diameter which is no less than an average diameter of the immune cell nucleus, passing a fluid containing the immune cell and the one or more exogenous substances through the plurality of pores to induce a mechanical stress to the immune cell and facilitate introduction of the one or more exogenous substances into the immune cell, and recirculating the fluid containing the immune cell and the one or more exogenous substances such that the same immune cell passes through the membrane more than once.

In one embodiment of the method as disclosed herein, the average pore diameter of the plurality of pores is no more than an average diameter of the immune cell.

In one embodiment of the method as disclosed herein, the specific flow rate for passing the fluid containing the immune cell and the one or more exogenous substances through the plurality of pores is from 10 nL/s to 1000 nL/s.

In one embodiment of the method as disclosed herein, the method further comprises, prior to the step of passing the fluid, activating the immune cell from an inactivated to an activated state by exposing the immune cell to one or more activation agents.

In one embodiment of the method as disclosed herein, the method is devoid of introducing one or more conditioning substances into the fluid after the activating step.

In one embodiment of the method as disclosed herein, the immune cell has not been pretreated to alter cell membrane pliability, prior to passing through the membrane.

In one embodiment of the method as disclosed herein, the method further comprises, prior to passing the immune cell through the membrane, seeding the immune cell into the fluid at a density of no less than 1 × 10⁵ cells/ml and adding the one or more exogenous substances into the fluid.

In one embodiment of the method as disclosed herein, the fluid containing the immune cell and the one or more exogenous substances has a volume of at least 20 mL.

In one embodiment of the method as disclosed herein, the fluid containing the immune cell and the one or more exogenous substances is substantially free of serum when the fluid is passed through the plurality of pores.

In one embodiment of the method as disclosed herein, the method further comprises providing a time interval of at least 5 minutes to rest the immune cell between any two consecutive passes through the membrane.

In one embodiment of the method as disclosed herein, the immune cell is a T cell.

In one embodiment of the method as disclosed herein, the T cell transfected with the one or more exogenous substances is to be used for immunotherapy.

In one embodiment of the method as disclosed herein, the one or more exogenous substances comprise a nucleic acid configured for expressing one or more exogenous genes for a therapeutic application.

In one embodiment of the method as disclosed herein, for each pass through the plurality of pores, the number of viable immune cells recovered after passing though the plurality of pores is at least 30% of the number of viable immune cells prior to passing through the plurality of pores.

In one embodiment of the method as disclosed herein, for each pass through the plurality of pores, the number of immune cells transfected with the one or more exogenous substances is at least 10% of the number of untransfected immune cells prior to passing through the plurality of pores.

In one embodiment of the method as disclosed herein, the number of immune cells transfected with the one or more exogenous substances increases with each additional pass across the membrane.

In one aspect, there is provided a system for introducing one or more exogenous substances into an immune cell, the system comprising, a membrane having a plurality of pores configured to allow the immune cell to pass through, wherein the plurality of pores has an average pore diameter which is no less than an average diameter of the immune cell nucleus, and a pump configured to pass a fluid containing the immune cell and the one or more exogenous substances through the plurality of pores to induce a mechanical stress to the immune cell and facilitate introduction of the one or more exogenous substances into the immune cell, wherein the pump is further configured to recirculate the fluid containing the immune cell and the one or more exogenous substances such that the same immune cell passes through the membrane more than once.

In one embodiment of the system as disclosed herein, the average pore diameter of the plurality of pores is no more than an average diameter of the immune cell.

In one embodiment of the system as disclosed herein, the system further comprises a vessel arranged to form an enclosed system such that the membrane is housed within the enclosed system.

### Definitions

The term “viable” as used herein refers to the ability of cells to perform one or more of the following functions: metabolism, growth, sustenance, and reproduction. The extent and character of these signs of viability may vary from one cell type to another, as known by those skilled in the art. Cell viability may be readily evaluated using techniques known to those skilled in the art. The term as used herein also refers to cells which are alive in the culture at a particular time.

The term “non-viable” as used herein refers to cells that are not capable of performing at least the following functions: metabolism, growth, sustenance, and reproduction, under any known conditions.

The term “substrate” as used herein is to be interpreted broadly to refer to any supporting structure.

The term “in vitro” refers to an artificial environment and to processes or reactions that occur within an artificial environment, or elsewhere outside a living organism. In vitro environments may include, but are not limited to, test tubes, cell cultures, bioreactors etc.

The term “in vivo” as used herein refers to the natural environment within the body of an organism (e.g., an animal or a human) and to processes or reaction that occur within a natural environment.

The term “cell culture” as used herein refers to any in vitro culture of cells.

The term “culture media” as used herein refer to media that are suitable to support the growth of cells of interest in vitro.

The term “micro” as used herein is to be interpreted broadly to include dimensions from about 1 micron to about 1000 microns. Exemplary sub-ranges that fall within the term include but are not limited to the ranges of from about 10 micron to about 900 microns, from about 20 micron to about 800 microns, from about 30 micron to about 700 microns, from about 40 micron to about 600 microns, from about 50 micron to about 500 microns, from about 60 micron to about 400 microns, from about 70 micron to about 300 microns, from about 80 micron to about 200 microns, or from about 90 micron to about 100 microns.

The term “nano” as used herein is to be interpreted broadly to include dimensions less than about 1000 nm. Exemplary sub-ranges that fall within the term include but are not limited to the ranges of less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, less than about 20 nm, or less than about 10 nm.

The term “particle” as used herein broadly refers to a discrete entity or a discrete body. The particle described herein can include an organic, an inorganic or a biological particle. The particle used described herein may also be a macroparticle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/- 5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01 %, 1.02% ... 4.98%, 4.99%, 5.00% and 1.1%, 1.2% ... 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a method and system for introducing/transfecting one or more exogenous substances into an immune cell are disclosed hereinafter.

In various embodiments, there is provided a method of introducing one or more exogenous substances into an immune cell, the method comprising, providing a membrane having a plurality of pores configured to allow the immune cell to pass through, wherein the plurality of pores has an average pore diameter which is no less than an average diameter of the immune cell nucleus, passing a fluid containing the immune cell and the one or more exogenous substances through the plurality of pores to induce a mechanical stress to the immune cell and facilitate introduction of the one or more exogenous substances into the immune cell, and recirculating the fluid containing the immune cell and the one or more exogenous substances such that the same immune cell passes through the membrane more than once. In various embodiments, the recirculating step comprises passing the same immune cell through the membrane in the same direction more than once, that is, the same immune cell passes through the membrane from a first surface and exits from a second surface of the membrane (i.e., the first and second surfaces being different surfaces) more than once. In various embodiments, the recirculation of the immune cells does not comprise allowing the immune cell through the membrane from the second surface to the first surface before subsequently passing the same immune cell from the first surface to a second surface (i.e., exiting from second surface). It will be appreciated that the cell referred to herein in the singular form includes plural meanings.

Advantageously, various embodiments of the method may be applicable to immune cells, for the purpose of engineering them for immunotherapy. Various embodiments of the method may be used for introducing/transfecting one or more exogenous substances such as foreign nucleic acid into immune cells to express exogenous genes for therapeutic applications. Advantageously, various embodiments of the method may be designed to be compatible with existing workflow for such preparations for clinical applications, including the use of appropriate media, flow conditions, and number of times cells are passed through a recirculating device (that is, a self-contained vessel in which the cells are circulated), in order to achieve high transfection without introducing new materials. It will be appreciated that an optimized set of conditions contributes to a high level of transfection.

In various embodiments, the step of recirculating the fluid containing the immune cell and the one or more exogenous substances comprises passing the same cell across the membrane for two or more times. In some embodiments, the cell is passed across the membrane twice, or thrice.

In various embodiments, the plurality of pores is configured to allow the cell to pass through while inducing a mechanical stress to the cell to facilitate the introduction of the one or more exogenous substances into the cell. In various embodiments, the mechanical stress comprises shear stress and/or deformation of the cell and/or cellular membrane. The mechanical stress may produce a transient perturbation in the cell membrane of the cell passing therethrough, which may increase the permeability of the cell membrane to facilitate introduction of the one or more exogenous substances. In various embodiments, the method relies solely on mechanical stress to introduce the one or more exogenous substances into the cell.

In various embodiments, the plurality of pores has pore dimensions that are substantially uniform across said plurality of pores. The substantially uniform pore dimensions may advantageously ensure that the mechanical stress applied to the cell is substantially consistent for each pore and across the plurality of pores.

In various embodiments, the pore dimensions are at least one of: average diameter, average cross-sectional area and average path length through the membrane. In various embodiments, the pore dimensions may be customized depending on the dimensions of the cell to be transfected. In various embodiments, the pore dimensions may be customized to achieve transfection, recovery and survivability of the cell. That is, the pore dimensions may be customized such that the cell undergoes an appropriate mechanical stress to achieve transfection of the one or more exogenous substances while passing through the pore and remains viable after passing through the pore.

In various embodiments, the plurality of pores define substantially linear paths through the membrane. In various embodiments, each of the substantially linear paths has a substantially uniform cross-sectional area throughout. In various embodiments, the substantially linear path has a cross-section with an average diameter that is capable of inducing the mechanical stress on the cell.

In various embodiments, the pore dimensions are in the micrometer range.

In various embodiments, the average pore diameter is from about 4 µm to about 20 µm, from about 5 µm to about 19 µm, from about 6 µm to about 18 µm, from about 7 µm to about 17 µm, from about 8 µm to about 16 µm, from about 9 µm to about 15 µm, from about 10 µm to about 14 µm, from about 11 µm to about 13 µm, from about 12 µm to about 13 µm, from about 21 µm to about 30 µm, from about 22 µm to about 29 µm, from about 23 µm to about 28 µm, from about 24 µm to about 27 µm, or from about 25 µm to about 26 µm.

In various embodiments, the plurality of pores is disposed in an area of the membrane that is accessible by said cell. In various embodiments, the diameter of the area of the membrane that is accessible by the cell may be in the range of from about 1 mm to about 100 mm, from about 5 mm to about 95 mm, from about 10 mm to about 90 mm, from about 15 mm to about 85 mm, from about 20 mm to about 80 mm, from about 25 mm to about 75 mm, from about 30 mm to about 70 mm, from about 35 mm to about 65 mm, from about 40 mm to about 60 mm, from about 45 mm to about 55 mm or from about 50 mm to about 55 mm. The flow resistance may also be substantially uniform across the area of the membrane that is accessible by the cells.

In various embodiments, the average path length of substantially linear paths ranges from about 10 µm to about 100 µm, from about 20 µm to about 90 µm, from about 30 µm to about 80 µm, from about 40 µm to about 70 µm, from about 50 µm to about 60 µm, from about 110 µm to about 150 µm, from about 120 µm to about 140 µm, or from about 120 µm to about 130 µm.

In various embodiments, the membrane has an average thickness from about 10 µm to about 100 µm, from about 20 µm to about 90 µm, from about 30 µm to about 80 µm, from about 40 µm to about 70 µm, from about 50 µm to about 60 µm, from about 11 0 µm to about 150 µm, from about 120 µm to about 140 µm, or from about 120 µm to about 130 µm.

In various embodiments, the plurality of pores has an average pore diameter which is no less than an average diameter of the cell nucleus. For example, the average pore diameter of the plurality of pores may be in the range of about 100% to 150% of the average diameter of the nucleus, about 105%, about 110%, about 115%, about 120%, about 125%, about 130%, about 135%, about 140%, about 145% or about 150% of the average diameter of the nucleus.

In various embodiments, the average pore diameter of the plurality of pores is no more than an average diameter of the cell. For example, the average pore diameter of the plurality of pores may be at least about 70% of the average cell diameter, at least about 75% of the average cell diameter, at least about 80% of the average cell diameter, at least about 85% of the average cell diameter, at least about 90% of the average cell diameter, at least about 95% of the average cell diameter, at least about 96% of the average cell diameter, at least about 97% of the average cell diameter, at least about 98% of the average cell diameter, or at least about 99% of the average cell diameter. In various embodiments, the average pore diameter of the plurality of pores may range from the average diameter of the cell nucleus to the average diameter of the cell, such that the average diameter of the cell nucleus sets the lower limit for the average pore size. For example, the average pore diameter may be between the average diameter of the cell nucleus and the average cell diameter. In various embodiments therefore, the average pore diameter is no less or no smaller or larger than the average diameter of the cell nucleus and no more or no larger or smaller than the average cell diameter.

In various embodiments, the average pore diameter is no more than about 100%, no more than about 105%, no more than about 110%, no more than about 115%, no more than about 120%, no more than about 125%, no more than about 130%, no more than about 135%, no more than about 140%, no more than about 145%, or no more than about 150% of the average diameter of the cell. In various embodiments, the average pore diameter of the plurality of pores is large enough to allow cells to pass through each pore in a single file manner. This may advantageously minimize the occurrence of clogging of the membrane.

In various embodiments, the step of passing the fluid containing the immune cell and the one or more exogenous substances across the membrane is performed at a specific flow rate. The fluid flow may be turbulent flow or laminar flow. The fluid flow may be turbulent or laminar flow prior to the immune cell and the one or more exogenous substances passing across the plurality of pores. The fluid flow may be turbulent or laminar flow after the immune cell and the one or more exogenous substances pass across the plurality of pores. The fluid flow may be turbulent or laminar flow while the immune cell and the one or more exogenous substances are passing across the plurality of pores. The immune cell and the one or more exogenous substances may pass across the plurality of pores at a substantially uniform speed. The immune cell and the one or more exogenous substances may pass across the plurality of pores at a non-uniform or fluctuating speed.

In various embodiments, the specific flow rate is from about 10 nL/s to about 45 nL/s, from about 15 nL/s to about 40 nL/s, from about 20 nL/s to about 35 nL/s, from about 25 nL/s to about 30 nL/s, from about 50 nL/s to about 100 nL/s, from about 55 nL/s to about 95 nL/s, from about 60 nL/s to about 90 nL/s, from about 65 nL/s to about 85 nL/s, from about 70 nL/s to about 80 nL/s, from about 75 nL/s to about 80 nL/s, from about 150 nL/s to about 1000 nL/s, from about 200 nL/s to about 950 nL/s, from about 250 nL/s to about 900 nL/s, from about 300 nL/s to about 850 nL/s, from about 350 nL/s to about 800 nL/s, from about 400 nL/s to about 750 nL/s, from about 450 nL/s to about 700 nL/s, from about 500 nL/s to about 650 nL/s, or from about 550 nL/s to about 600 nL/s.

In various embodiments, the step of passing the fluid containing the immune cell and the one or more exogenous substances across the membrane is performed at a predetermined average flow velocity. In various embodiments, the average flow velocity is estimated by the volumetric flow rate divided by the cross-sectional area of pores. For example, for a membrane having 10,000 pores per cm², with each pore measuring about 12-microns (µm) in diameter, the total pore area (in square microns) is estimated by multiplying the membrane area (in cm²) by the number of pores per cm² (i.e., 10,000 pores/cm²) by the area of each pore (Π × (12/2)²). In various embodiments, the average pore size is chosen such that the average pore diameter is larger than the average nucleus diameter of the immune cell, but smaller than the average cell plasma membrane diameter of the immune cell. In various embodiments, the flow velocity is empirically determined for different cell types having different tolerance for shear stresses.

In various embodiments, the method further comprises, prior to the step of passing the fluid through the membrane, activating the immune cell from an inactivated state to an activated state by exposing the immune cell to one or more activation agents. In various embodiments, the method comprises, prior to the step of passing the fluid through the membrane, activating the immune cell from a quiescent/ resting state to an activated state by exposing the immune cell to one or more activation agents. The immune cells may be NK (natural killer) cells, macrophages, neutrophils, eosinophils, B cells or T cells. The immune cells may be from a lymphocyte cell line. Lymphocytes may include T cells, B cells and NK cells. In some examples, the immune cell may be a T lymphocyte or an NK (natural killer) cell., both of which may be activated and/or expanded ex vivo. In some examples, the T lymphocyte or NK cell is an autologous T lymphocyte or an autologous NK cell isolated from a patient (e.g., a human patient). In some examples, the T lymphocyte or NK cell is an allogenic T lymphocyte or an allogenic NK cell. The T lymphocyte may be activated in the presence of one or more activation agents selected from the group consisting of anti-CD3 antibody, anti-CD28 antibody, IL-2, and phytohemoagglutinin. The NK cell may be activated in the presence of one or more activation agents selected from the group consisting of CD 137 ligand protein, CD 137 antibody, IL15 protein, IL-15 receptor antibody, IL-2 protein, IL-12 protein, IL-21 protein, and K562 cell line. In one embodiment, the immune cell is a T cell. In various embodiments, the T cell transfected with the one or more exogenous substances is to be used for immunotherapy. Immunotherapy may include TCR-T (T-cell receptor therapy) and CAR-T (chimeric antigen receptor therapy).

It will be appreciated that the method as disclosed herein may be used for introducing one or more exogenous substances into other types of cells. The types of cells which may be suitable for use with the method may include animal cell, bacterial cell, protozoal cell, mammalian cell or human cell. In various embodiments, the cell may be derived from a primary cell line. In various embodiments, the cell may be a mammalian cell. In various embodiments, the cell may be a human cell. In various embodiments, the membrane for introducing one or more exogenous substances into a cell is adapted to work for a cell which does not have a cell wall. For example, in various embodiments, the method may not be applicable for cells with cell walls such as most plant cells and fungal cells and a large proportion of bacterial cells.

In various embodiments, the step of activating the immune cell comprises culturing the immune cell in vitro in the presence of the one or more activation agents for a period from about 1 day to 7 days, from about 2 days to 6 days, from about 3 days to 5 days, or from about 3 days to 4 days.

In various embodiments, the method further comprises, subsequent to the step of activating the immune cell, a step of expanding/proliferating the immune cell in vitro to increase the number of activated immune cells. Expanding in vitro may involve any method that results in an increase in the number of activated immune cells. In various embodiments, the step of expanding the immune cell comprises culturing the immune cell in vitro for a period from about 1 day to 7 days, from about 2 days to 6 days, from about 3 days to 5 days, or from about 3 days to 4 days.

One example of a T cell stimulation and expansion protocol is described in Trickett A, Kwan YL. T cell stimulation and expansion using anti-CD3/CD28 beads. J Immunol Methods. 2003 Apr 1 ;275(1-2):251-5, the disclosure of which is completely incorporated herein by reference. Trickett A et al describes a method for stimulating T lymphocytes using magnetic beads coated with anti-CD3 and anti-CD28. In various embodiments, tests of immune cell function such as proliferation assay and cytotoxicity assay may be performed on the immune cells after activation or proliferation.

In various embodiments, the method further comprises, subsequent to the step of expanding the immune cell, a step of dissociating the immune cells to single cells through trypsinization, mechanical dislodgement, or any common method known in the art. It will be appreciated that the step of dissociating the cells may be skipped if the cell is a non-adherent cell.

In various embodiments, the immune cell has not been pretreated to alter cell membrane and/or nuclear membrane pliability, prior to passing through the membrane comprising a plurality of pores. Pretreatment which alters cell membrane and/or nuclear membrane pliability may include but are not limited to chemical pretreatment, drug pretreatment, electrical pretreatment and the like. For example, drug pretreatment may involve treating the immune cell with one or more drugs that result in partial disassembly of the cell and/or nuclear membrane. Such pretreatment typically ‘softens’ the nucleus and allows the cell to pass through the pore more easily. In various embodiments, the fluid containing the immune cell and the one or more exogenous substances is substantially devoid of a softening agent that alters cell membrane and/or nuclear membrane pliability. In certain applications, such as clinical applications, the addition of any component, such as drugs used for ‘softening’ the nucleus is to be avoided, since it will require excessive testing to get through regulatory approval. Advantageously, by not pretreating the immune cell with chemicals or drugs, various embodiments of the method are compatible with, and can be used for cell-based therapeutics that require introduction into the body (e.g., human or animal body).

In various embodiments, the method further comprises, prior to passing the cell through/across the membrane, seeding the immune cell into the fluid with a cell density of no less than 1 × 10⁵ cells/ml, no less than 2 × 10⁵ cells/ml, no less than 4 × 10⁵ cells/ml, no less than 6 × 10⁵ cells/ml, no less than 8 × 10⁵ cells/ml, no less than 1 × 10⁶ cells/ml, no less than 2 × 10⁶ cells/ml, no less than 3 × 10⁶ cells/ml, no less than 4 × 10⁶ cells/ml, or no less than 5 × 10⁶ cells/ml. In various embodiments, the cells are suspended in the fluid. In various embodiments, a higher seeding density may advantageously improve transfection efficiency.

In various embodiments, the method further comprises, prior to passing the cell through/across the membrane, a step of adding the one or more exogenous substances into the fluid. In various embodiments, the one or more exogenous substances may also be referred to as the payload or cargo of interest. In various embodiments, the one or more exogenous substances is added into the fluid (i) prior to the stage of activating the immune cells, (ii) at or during the stage of activating the immune cells, and/or (iii) after the stage of activating the immune cells. In some embodiments, the one or more exogenous substances is added to the fluid after the stage of activating the immune cells. The one or more exogenous substances that are to be introduced into the immune cell is selected from the group consisting of small molecules, nanoparticles, macromolecules, oligonucleotides, plasmids, RNA, DNA, peptides, proteins, compositions of matter and combinations thereof. In various embodiments, the one or more exogenous substances is a nucleic acid. In various embodiments, the nucleic acid is mRNA. In various embodiments, the mRNA is a modified mRNA. Various embodiments of the device and method disclosed herein are configured/arranged to introduce modified mRNA rather than DNA as cargo into the cell. In various embodiments, the method is devoid of introducing one or more substances into the fluid after the activating step. In various embodiments, the method is devoid of introducing substances apart from the payload or cargo of interest (i.e., the one or more exogenous substances such as a nucleic acid) into the fluid after the activating step. For example, the method may be devoid of introducing conditioning substances for conditioning the fluid (e.g., culture medium, cytokines, stimulants etc.) into the fluid after the activating step. The method may also be devoid of introducing additional cells/organisms (e.g., immune cell etc.) into the fluid after the activating step. By avoiding introduction of new molecules apart from the payload into the workflow, the method may avoid having to perform extensive safety studies when used in clinical applications e.g., immunotherapy.

In various embodiments, the fluid containing the immune cell and the one or more exogenous substances has a volume of at least 20 mL, at least 25 mL, at least 30 mL, at least 35 mL, at least 40 mL, at least 45 mL, at least 50 mL, at least 55 mL, at least 60 mL, at least 65 mL, at least 70 mL, at least 75 mL, at least 80 mL, at least 85 mL, at least 90 mL, at least 95 mL, or at least 100 mL. Advantageously, in various embodiments, the fluid containing the immune cell and the one or more exogenous substances has a working volume of at least about 20 mL which is a practical amount for cell therapy use.

In various embodiments, the fluid containing the immune cell and the one or more exogenous substances is a media e.g., culture media. In various embodiments, the fluid comprises a low-serum or serum-free media. In various embodiments, the fluid originally comprise serum (e.g., when culturing and/or activating the immune cells in accordance with established protocols) but the serum may be substantially removed from the fluid prior to passing the fluid through the membrane. In various embodiments, the fluid containing the immune cell and the one or more exogenous substances is substantially free of serum when the fluid is passed through the plurality of pores. A low-serum or serum-free media may advantageously improve transfection efficiency because the relatively low level of, or absence of serum may minimize degradation of the one or more exogenous substances e.g., mRNA.

In various embodiments, the method further comprises providing a time interval of at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, at least 120 minutes, at least 125 minutes, at least 130 minutes, at least 135 minutes, at least 140 minutes, at least 145 minutes, at least 150 minutes, at least 155 minutes, at least 160 minutes, at least 165 minutes, at least 170 minutes, at least 175 minutes, or at least 180 minutes between any two consecutive passes across the membrane e.g. to rest the cell. In various embodiments, the time interval between any two consecutive passes across the membrane may advantageously aid in the survivability of the cell as the cell is allowed to rest before the next pass.

In various embodiments, the cell remains in a viable form after passing through the membrane via the plurality of pores. In various embodiments, for each pass through the plurality of pores, the number of viable immune cells recovered after passing though the plurality of pores is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the number of viable immune cells prior to passing through the plurality of pores. In various embodiments, cell recovery rate is defined as a percentage of the number of viable cells that have passed through the plurality of pores relative to the original number of viable cells prior to passing through the plurality of pores. In various embodiments, the cell recovery rate may be determined by cell counting techniques known in the art. In various embodiments, the percentage of unrecovered cells for each pass may be substantially constant.

In various embodiments, for each pass through the plurality of pores, the number of immune cells transfected with the one or more exogenous substances is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the number of untransfected immune cells prior to passing through the plurality of pores. In various embodiments, transfection efficiency is defined as a percentage of the number of cells transfected with the one or more exogenous substance relative to the original number of untransfected cells. In various embodiments, transfection efficiency may be determined by techniques known in the art such as FACS (fluorescence activated file sorting).

In various embodiments, the method is capable of achieving increasingly higher transfection efficiency with each additional pass of the cell through the membrane. The number of immune cells transfected with the one or more exogenous substances increases with each additional pass across the membrane. It will be appreciated that while the transfection efficiency may increase with the use of smaller pore sizes in the plurality of pores, cell death also increases. Various embodiments of the method balance these two factors, by choosing an average pore diameter that is larger than the cell nucleus (thereby reducing cell death), while achieving high stress.

In various embodiments, there is provided a method of cell transfection that (a) relies solely on resultant mechanical shear stress from passing cells through pores in a filter, to induce the uptake of nucleic acids; (b) does not require the addition of media and can take place in a single container; (c) passes cells through pores that are larger than the diameter of the cell nucleus; and (d) passes cells through pores multiple times with intervals between each pass, and with transfection efficiency increasing with each pass.

In various embodiments, there is provided a system for introducing one or more exogenous substances into an immune cell, the system comprising, a membrane having a plurality of pores configured to allow the immune cell to pass through, wherein the plurality of pores has an average pore diameter which is no less than an average diameter of the cell nucleus, and a pump configured to pass a fluid containing the immune cell and the one or more exogenous substances through the plurality of pores to induce a mechanical stress to the immune cell and facilitate introduction of the one or more exogenous substances into the immune cell, wherein the pump is further configured to recirculate the fluid containing the immune cell and the one or more exogenous substances such that the same immune cell passes through the membrane more than once.

Various embodiments of the system disclosed herein do not utilize a microfluidic system. Instead, various embodiments of the system utilize a membrane-based system that is self-contained, and designed for high-throughput, high efficiency, and low-cost preparation of cells. Various embodiments of the system are capable of achieving high transfection, which is uniquely challenging for immune cells, e.g., lymphocytes which have small cytoplasmic volumes, by reducing the pore size but limiting it to at least the nucleus diameter, by increasing cell numbers, and increasing the number of passes through the membrane. Various embodiments of the membrane disclosed herein comprise a plurality of pores that are larger than an average diameter of the cell nucleus. In various embodiments, the membrane as disclosed herein comprises a plurality of pores that are larger than an average diameter of the entire cell. Various embodiments of the system are configured/arranged to allow the cells to recirculate through the plurality of pores of the membrane multiple times.

In various embodiments, the membrane comprises a first surface for allowing fluid to enter the plurality of pores and a second opposite surface for allowing fluid to exit the plurality of pores. In various embodiments, the first and second surfaces have a two-dimensional shape which includes, but is not limited to, circular, elliptical, and polygonal shapes such as triangle, square, pentagon, hexagon, heptagon, octagon, and the like. In one embodiment, the first and second surfaces have a circular shape.

In various embodiments, the membrane comprises a polymer. In various embodiments, polymer materials that are suitable for fabricating the membrane include but are not limited to polyimide, polyester, polycarbonate, polyethylene, polyethersulfone, perflourinated cyclobutane, polymethylmethacrylate (PMMA), various photoresists, parylene, and other polymers.

In various embodiments, the system further comprises a vessel/container arranged to form an enclosed system such that the membrane is housed within the enclosed system. In various embodiments, the membrane is housed within a single container such as a blood bag and the enclosed system does not require change of media. In various embodiments, the mixture does not require addition or change of media and can take place in a single container.

In various embodiments, the system further comprises a housing/casing for holding the membrane. The housing may comprise a first opening and a second opening disposed at substantially opposite ends of the housing. The first opening may be an inlet for receiving fluid e.g., fluid containing the cell and the one or more exogenous substances to be delivered and the second opening may be an outlet for allowing fluid to exit from the housing. The housing may further comprise an internal chamber for holding the membrane. The membrane may extend across the cross section of the internal chamber, dividing the internal chamber into a first sub-chamber and a second sub-chamber, the first and second sub-chambers being in fluid communication with each other. The first surface of the membrane may be positioned to face the first opening (i.e., inlet) for allowing fluid to enter the plurality of pores and the second opposite surface of the membrane may be positioned to face the second opening (i.e., outlet) for allowing fluid to exit the plurality of pores.

In various embodiments, the housing may comprise one or more protrusions disposed on the inner surface of the housing for supporting and maintaining the membrane in a fixed position. The membrane may be further secured to the housing using sealing means to seal the periphery of the membrane such that fluid is directed to flow through the plurality of pores and not around the periphery of the membrane. Sealing means may comprise using polydimethylsiloxane (PDMS), nitrocellulose, epoxy, cyanoacrylates, silicone, and other adhesives or the like.

In various embodiments, the housing may be made from polymers which include but are not limited to polytetrafluoroethylene, polysulfone, polyethersulfone, polypropylene, polyethylene, fluoropolymers, cellulose acetate, polystyrene, polystyrene/acrylonitrile copolymer, PVDF, and combination thereof. It would be appreciated that the housing may advantageously possess qualities such as low material cost, ease of fabrication, ease of mass production, sterilisable, low toxicity to cells etc.

In various embodiments, the system comprises a pump for passing a fluid, e.g., fluid containing the cell and the one or more exogenous substances, through the plurality of pores of the membrane. In various embodiments, the pump is in fluid communication with the membrane. In various embodiments, a pump is a device that moves fluids (liquid or gas) by mechanical action. In various embodiments, the pump is configured to recirculate the cell such that the same cell passes through the membrane more than once.

In various embodiments, the pump is a peristaltic pump. In various embodiments, the pump is configured to pass the cell and the one or more exogenous substance through each of the plurality of pores at a flow rate of from about 10 nL/s to about 45 nL/s, from about 15 nL/s to about 40 nL/s, from about 20 nL/s to about 35 nL/s, from about 25 nL/s to about 30 nL/s, from about 50 nL/s to about 100 nL/s, from about 55 nL/s to about 95 nL/s, from about 60 nL/s to about 90 nL/s, from about 65 nL/s to about 85 nL/s, from about 70 nL/s to about 80 nL/s, from about 75 nL/s to about 80 nL/s, from about 150 nL/s to about 1000 nL/s, from about 200 nL/s to about 950 nL/s, from about 250 nL/s to about 900 nL/s, from about 300 nL/s to about 850 nL/s, from about 350 nL/s to about 800 nL/s, from about 400 nL/s to about 750 nL/s, from about 450 nL/s to about 700 nL/s, from about 500 nL/s to about 650 nL/s, or from about 550 nL/s to about 600 nL/s.

In various embodiments, the system comprises a tubing assembly for connecting the pump to the housing/casing for holding the membrane such that fluid, e.g., fluid containing the cell and the one or more exogenous substances, is allowed to recirculate within the system. In various embodiments, the system is an enclosed recirculating system.

In various embodiments, the flow resistance and flow rate are substantially uniform across the area of the membrane that is accessible by said cell.

In various embodiments, there is provided a membrane, method and system comprising the following features: (a) applying purely mechanical shear stress on cells passing through pores in a filter device to induce uptake of one or more substances e.g. nucleic acids including mRNA into cells e.g. immune cells, trypsinized cells or nonadherent cells; (b) achieving transfection in a single container, such as a blood bag, without needing changes of media; (c) passing cells through multiple pores larger than the cell nucleus’ diameter, in order to improve cell recovery and survival; (d) passing cells through the pores multiple times, with intervals intended to aid cell survival; and (e) increasing transfection efficiency with each pass through the pores.

### BRIEF DESCRIPTION OF FIGURES

FIG. 1A is a schematic drawing of a deformation-based transfection showing a cell prior to passing through a pore of a membrane in an example embodiment.

FIG. 1B is a schematic drawing of the deformation-based transfection showing the cell while passing through the pore of the membrane in the example embodiment.

FIG. 1C is a schematic drawing of the deformation-based transfection showing the cell after passing through the pore of the membrane in the example embodiment.

FIG. 2 is a micrograph of a lymphocyte in an example embodiment.

FIG. 3A is a schematic drawing of a deformation-based transfection showing a membrane with a pore size smaller than the diameter of a cell nucleus in an example embodiment.

FIG. 3B is a schematic drawing of a deformation-based transfection showing a membrane with a pore size that is at least the diameter of a cell nucleus in an example embodiment.

FIG. 4A is a typical FACS (fluorescence-activated cell sorting) result for Jurkat cells transfected with eGFP mRNA, analyzed two days after passing through the presently disclosed system once (46% transfection efficiency) in an example embodiment.

FIG. 4B is a typical FACS result for Jurkat cells transfected with eGFP mRNA, analyzed two days after passing through the presently disclosed system twice (77% transfection efficiency) in an example embodiment.

FIG. 5A is a brightfield micrograph of cells after transfection in an example embodiment.

FIG. 5B is a fluorescence micrograph of cells after transfection in the example embodiment.

FIG. 6 is a chart showing cell transfection efficiency as determined by FACS for different number of passes of cells through a membrane in an example embodiment.

FIG. 7A is a chart showing cell recovery after passing through membranes in an example embodiment.

FIG. 7B is a chart showing cell recovery as a percentage of previous pass in the example embodiment.

FIG. 8A is a schematic diagram of a system for introducing one or more exogenous substances into a cell in an example embodiment.

FIG. 8B is a photograph of the system for introducing one or more exogenous substances into a cell in the example embodiment.

FIG. 9 is a graph showing changes in cell diameters of primary T-cells cultured in AIM/V and 501U/mL or 200IU/mL IL-2 with time in an example embodiment.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural and chemical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

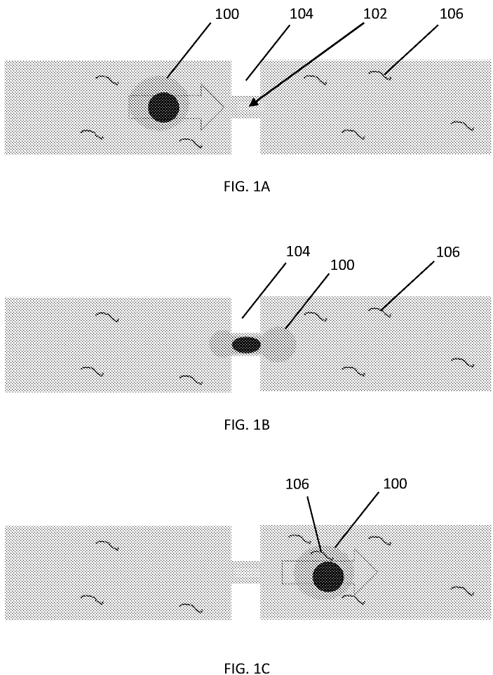

FIG. 1A to FIG. 1C are schematic drawings of a deformation-based transfection. The inventors had previously demonstrated a device and method of transfection (PipFect) using shear stress for various cell lines. The putative mechanism for this transfection is that the cells undergo mechanical stress during the deformation, which temporarily disrupts its nuclear membrane, resulting in uptake of a payload.

Briefly, cells 100 were pre-treated with a drug to allow partial disassembly of the nuclear membrane, making them more pliable prior to passing through a pore 102 of a membrane 104 (FIG. 1A). This ensures that the cells can deform through the pores, which may be smaller than the nuclear diameter. The cells 100 were then mixed with a payload 106 of interest (typically plasmid DNA encoding a reporter gene) and passed through micron-sized pores to deform them (FIG. 1B), and directly into serum-containing media (FIG. 1C). After a stipulated amount of time, the cells were characterized by the expression of the reporter. Pretreatment with drugs allows the use of smaller pores to achieve higher stresses and improve transfection efficiency (FIG. 1C).

The inventors noted that immunotherapy e.g., T-cell therapy such as Chimeric Antigen Receptor-T cell (CAR-T) therapy and/or T-Cell Receptor (TCR) therapy is a possible application of the above technology. However, several aspects of PipFect are not compatible with cell therapy.

Firstly, to be used in actual clinical application, the addition of any component, such as the drugs used for ‘softening’ the nucleus, is to be avoided, since it will require excessive testing to get through regulatory approval.

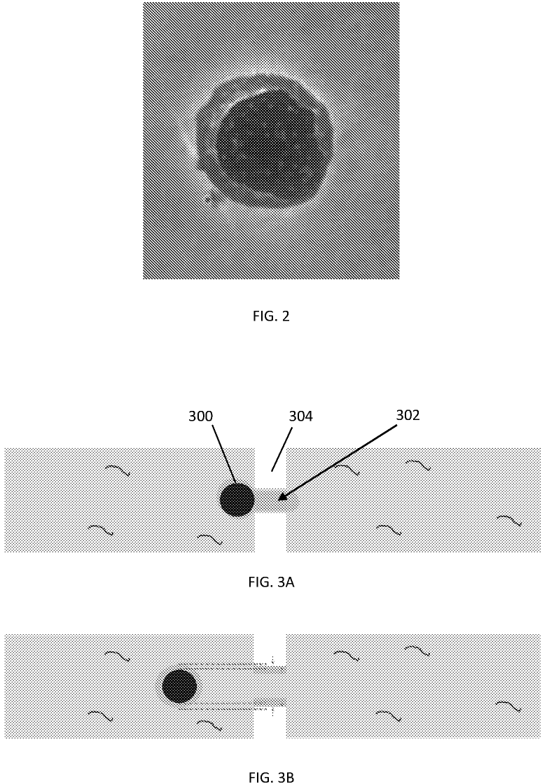

Secondly, lymphocytes have much smaller cytoplasm volumes than other cells, such as HeLa, HEK, and CHO. As shown in FIG. 2, lymphocytes, such as Jurkats, have very small cytoplasmic volumes, relative to their nuclear volume. Without the drugs to ‘soften’ up the nucleus, passing these cells through the pores can result in nucleus rupture, and consequently, cell death. This presents a challenge, since it is unclear from the outset whether a suitable pore size and shear condition can be found, that achieves high transfection efficiency, and cell recovery.

Thirdly, the form factor of the previous method PipFect was a pipette tip, through which a small volume (typically about 100 µL to about 200 µL) of cell/payload mixture was pushed, using a micropipettor. Not only is this volume too small to be practical for cell therapy use (around about 20 mL), the micropipettor is also unable to provide good control over the flow rate, which is recognised to play a role in controlling the transfection efficiency.

Fourthly, the PipFect device also requires culture to be conducted on a plate, when it may be preferable to perform the culture inside a self-contained vessel.

Example embodiments of the method and system as disclosed herein seek to overcome the abovementioned problems associated with the PipFect device and method.

In particular, example embodiments of the method and system seek to optimize the pore size through which a cell passes. As shown in FIG. 3A, in the absence of drugs that cause partial disassembly of the nuclear membrane, the cell nucleus of a cell 300 remains stiff, and may not pass through a pore 302 of a membrane 304 easily, especially if the pore size is smaller than the diameter of the cell nucleus. The result is that the cells can either be physically filtered out of the media (which will result in clogging of the device), or pass through the pores, and be severely damaged, to the point of cell death, in the process of forced deformation. As shown in FIG. 3B, the presently disclosed method and system emphasize on both cell recovery and transfection efficiency without the use of drugs that cause partial disassembly of the nuclear membrane. As such, the pore size is relatively larger, and the diameters of the cell nucleus and the entire cell (including small volume of cytoplasm) may be used to place upper and lower limits on the pore size.

### EXAMPLE

### Transfection of Lymphocyte Cell Line

Various embodiments of the cell transfection technology disclosed herein are capable of transfecting cells obtained from a lymphocyte cell line e.g., Jurkat cells.

The typical process for nonviral gene transfection involves the use of electroporation, which requires the cells to be removed from the expansion/stimulation media and placed in a low conductivity buffer for treatment in a specialized instrument capable of pulsing the cells in an electric field.

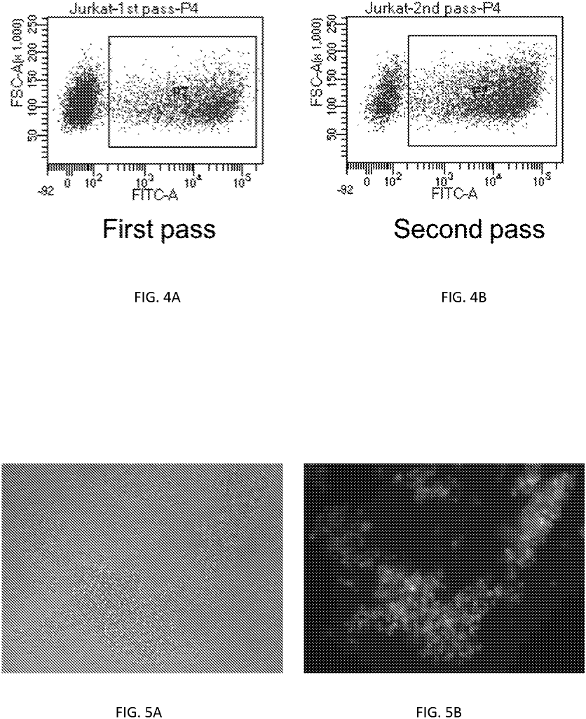

Various embodiments of the cell transfection technology disclosed herein seeks to simplify the workflow for cell transfection, by removing these handling steps. In this example, the payload (in this case, messenger RNA, or mRNA) is added to the cell mixture, directly into a low-serum or serum-free media, and the mixture is passed through a filtration device (flow rate from about 50nL/s to about 100 nL/s through each pore). After a fixed interval (typically about 2 hours), the same cells are passed through the membrane again. It is noted that the efficiency increases significantly on the second pass (see FIG. 4B). This process can be repeated several times, though for Jurkat cells, the increase in transfection efficiency was found to be almost two-fold after the second pass, but only marginally on subsequent passes.

It will be appreciated that these parameters (i.e., interval, number of passes, pore diameter, etc) depend on the type, state, and number of cells treated. Nevertheless, since Jurkats are a T lymphocyte cell line, the parameters here may serve as a good starting point for other similar cells, especially for T-cell engineering in immunotherapy applications.

It will also be appreciated that control of the various parameters allows for good transfection, as well as cell recovery, which are typically mutually exclusive goals. The inventors accomplished these by choosing a pore size that is larger than the cell nucleus diameter, which would normally mean minimal stress on the cell. However, the inventors then subjected the cells to around 5 to 10 times the flow rate at the pores than previously attempted.

The inventors showed that it is possible to achieve these goals of transfection efficiency and cell recovery through judicious choice of the membrane pore size (e.g., 12 microns) and density (e.g., 1E4/cm²). The inventors were also able to demonstrate that cells passed through the membrane multiple times, without introducing additional mRNA, can yield higher transfection efficiency. FIG. 4A and FIG. 4B show typical transfection results, in which Jurkat cells are transfected with eGFP green fluorescence protein, analyzed by FACS. FIG. 5A and FIG. 5B show that the transfected cells were highly-fluorescent in the micrographs. The brightfield (FIG. 5A) and fluorescence (FIG. 5B) images show that the transfection efficiency is very high in the sample. Quantitation by FACS indicate around 77% transfection efficiency.

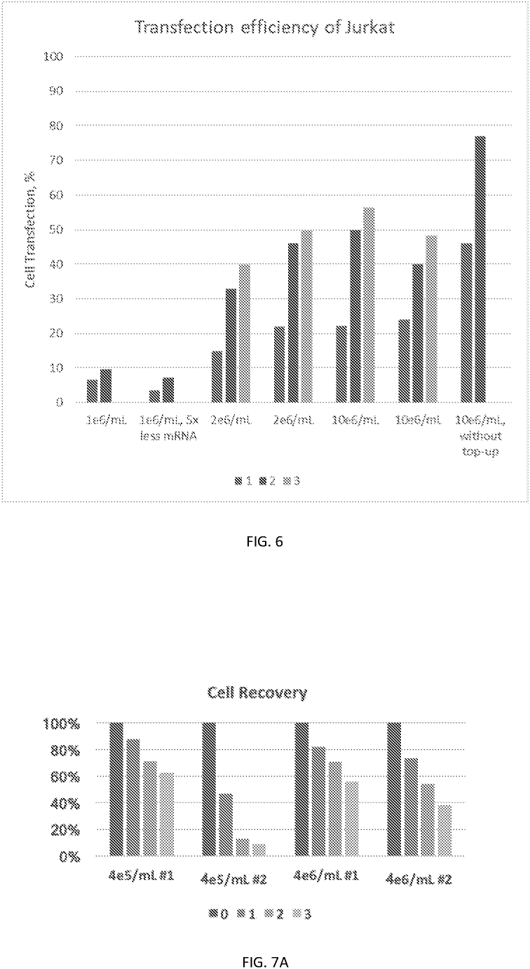

Transfection efficiency generally improves with the number of cells in the mixture (see FIG. 6). Furthermore, while transfection efficiency improved significantly between one and two passes through the filter, there was less improvement with subsequent passes. The improvement achieved by increasing from one to two passes is significantly greater than from two to three passes. It was also found that if the cells are left in OptiMEM after transfection, without topping up with RPMI 1640 media containing serum, the efficiency is further improved to 77% (see last two columns of FIG. 6).

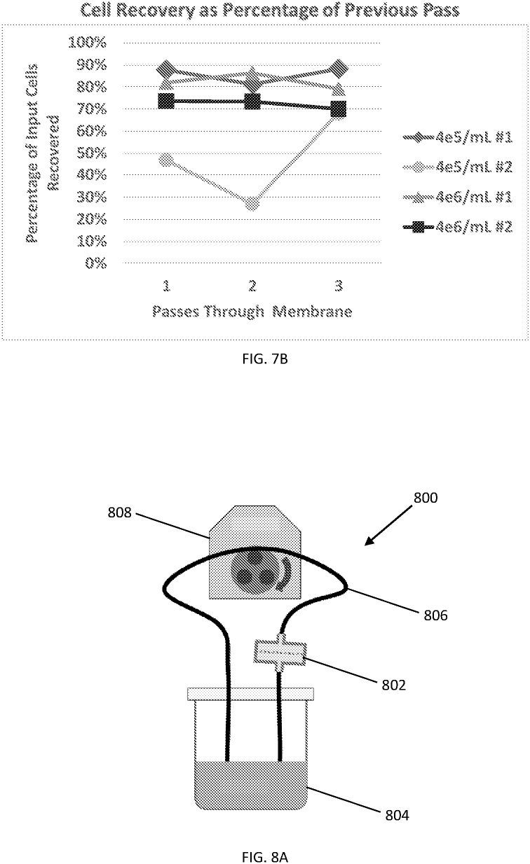

Although multiple passes improve transfection, they also reduce the number of cells recovered. As shown in FIG. 7A, more cells are lost with each pass through the membrane. However, the proportion of cells lost with each pass appears to be similar (see FIG. 7B). The cell recovery appears to be quite similar regardless of the cell concentration used.

The manufactured tips have some variability in the open aperture. This is because the membranes are manually glued onto the holders, and the glue can impinge on the aperture. This variability can be seen in the cell recovery results (FIG. 7B, series labeled “4e5/mL #2”). It is believed that by changing the assembly method, using pre-cut membranes clamped in fabricated holders, this variability can be greatly reduced.

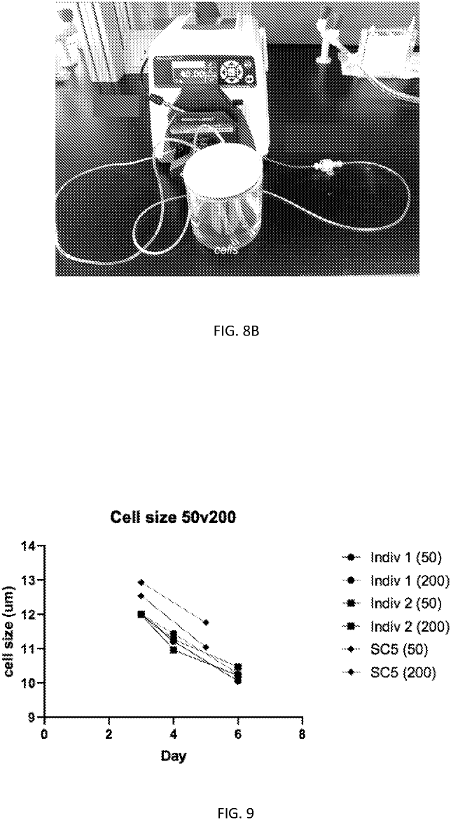

The inventors have also tested re-circulating the media and buffer in an enclosed vessel, using a peristaltic pump. FIG. 8A and FIG. 8B show a system 800 for introducing one or more exogenous substances into a cell in an example embodiment. The system 800 comprises a membrane device 802 according to embodiments as described herein coupled to a vessel 804 via a tubing assembly 806. In the example embodiment, the system 800 is a self-contained, closed loop circulating system. The vessel 804 is configured to contain a suspension of cells in a suitable media and buffer. The vessel 804 comprises an outlet for allowing fluid e.g., cell suspension to leave the vessel and an inlet for allowing fluid to enter the vessel via the tubing assembly 806. A pump e.g., peristaltic pump 808 is provided to circulate the cell suspension through the membrane device 802 and the tubing assembly 806. A portion of the tubing assembly 806 is disposed within a head of the peristaltic pump 808 such that a rotor of the peristaltic pump 808 can operate against the tubing assembly 806 for pumping the cell suspension through the tubing assembly 806 and the membrane device 802. In the example embodiment, the cell suspension may be recirculated through the system 800 such that each cell in the cell suspension passes through the membrane device 802 at least once.

The inventors have verified that the flow rates needed can be achieved using such a system. The applicability of the principles derived herein may be translated to a larger, recirculating system.

In summary, the inventors have demonstrated a method to perform transfection that result in high transfection (more than 75% transfection efficiency), as well as cell recovery (about 70% of cells recovered after two passes). This simple to use method can be performed in a closed, re-circulating system that requires minimum handling, and uses only cheap instrumentation, such as a peristaltic pump, to accomplish the desired gene transfection.

### Transfection of Primary T-Cells

Various embodiments of the cell transfection technology disclosed herein are capable of transfecting cells obtained from a primary cell line e.g., primary T-cells. Unlike Jurkats (about 11-13 microns), primary T-cells have very small cell diameters when first harvested (about 6-7 microns). However, the cells typically receive various treatments in culture prior to transfection, and the inventors closely followed established protocols.

Firstly, primary T-cells have to undergo activation for 3 days, prior to expansion in vitro, and this is part of the protocol for preparation of immune cells for immunotherapy. It will be appreciated that this process has profound effects on the cell diameters. FIG. 9 is a graph showing changes in the cell size of primary T-cells cultured in AIM/V and 501U/mL or 200IU/mL IL-2 over time. As shown, there is gradual decrease in cell diameter with time. Cells were activated on Day 0 with CD3/CD28. The inventors followed reported protocols and transfected the cells 4 days after activation. It will be appreciated that this protocol is subjected to change, depending on the specific protocol used.

Secondly, since the cells have a diameter of around 11 microns, a membrane with 10-micron pores was used. This pore size is slightly larger than the cell nucleus diameter (about 9 microns), but slightly smaller than the cell diameter. The same experiment was performed with cells that have diameters of 8 and 9 microns.

Table 1 provides a summary of the pore size, culture media conditions and transfection efficiency achieved using embodiments of the device and method disclosed herein.

TABLE 1

Summary of pore size, culture media conditions and transfection efficiency achieved using the presently disclosed device and method.

| Size | Media | Efficiency |
|---|---|---|
| 11 | AIM-V, 2% AB Serum, IL-2* | 0% |
| 11 | OPTIMEM | 49.9% |
| 11 | AIM-V, IL-2 | 34.3% |
| 9 | OPTIMEM | 14.9% |
| 9 | AIM-V, IL-2 | 20.8% |
| 8 | OPTIMEM | 3.4% |
| 8 | AIM-V | 2.3% |

* This is the media condition the cells are cultured under in a clinical protocol.

Further optimization to the protocol may be carried out while still allowing the primary T-cells to be efficiently transfected. One principle adhered to in this process is that while existing components to which the cells are exposed may be removed, new molecules will not be introduced into the workflow, so as to avoid having to perform extensive safety studies. Using the media that the cells are known to have been cultured in (AIM-V, 2% AB Serum, IL-2) for clinical applications, the inventors noted that there was almost no transfection. However, this is mostly attributed to the presence of serum, which is known to degrade mRNA, which is the payload. By removing the serum, much better transfection efficiency was achieved.

In summary, the inventors observed that relative to the pore size, the smaller the cells, the worse the transfection efficiency. This is expected, since the stresses to which the cells are subjected increases with larger cell sizes. However, the trade-off for cell production is that the cell numbers recovered is lower when the diameter is too large with respect to the pore size. Therefore, the two factors of transfection efficiency and cell recovery should be balanced.

### APPLICATIONS

Embodiments of the methods disclosed herein provide a method and system of introducing one or more exogenous substances into an immune cell. In various embodiments, the method and system are applicable to immune cells, for the purpose of engineering them for immunotherapy.

In various embodiments, the cell transfection technology disclosed herein is an improvement of previous work done on cell transfection. It was previously demonstrated the transfection of cells using a combination of drugs and mechanical shearing, can achieve reasonably good results for many cell lines. However, the drugs used, culture conditions, and the workflow are not compatible with cell therapy standards. By referring to parameters reported in the literature for preparation of patient immune cells used in immunotherapy e.g., T-cell therapy such as Chimeric Antigen Receptor-T cell (CAR-T) therapy and/or T-cell receptor (TCR) therapy, the inventors developed a new protocol that works very well with the immune cell line (e.g., Jurkat), with transfection efficiency exceeding 75%, and which requires minimal handling of the cells. Furthermore, unlike the previous work, cell recovery and survival are actively pursued in the presently disclosed cell transfection technology. The inventors have also carefully tested a range of conditions, including cell concentration, membrane pore size, pore density, and flow rate.

Advantageously, embodiments of the method and system disclosed herein are designed to be compatible with existing workflow for such preparations for clinical applications, including the use of appropriate media, flow conditions, and number of times cells are passed through a recirculating device (that is, a self-contained vessel in which the cells are circulated), in order to achieve high transfection without introducing new materials. It will be appreciated that these high levels of transfection may be achieved by embodiments of the method and system disclosed herein.

Advantageously, various embodiments of the method and system utilize a membrane-based system that is self-contained, and designed for high-throughput, high efficiency, and low-cost preparation of cells. Various embodiments of the device are capable of achieving high transfection, which is uniquely challenging for lymphocytes which have small cytoplasmic volumes, by reducing the pore size but limiting it to at least the nucleus diameter, by increasing cell numbers, and increasing the number of passes through the membrane device.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of introducing one or more exogenous substances into an immune cell, the method comprising,
providing a membrane having a plurality of pores configured to allow the immune cell to pass through, wherein the plurality of pores has an average pore diameter which is no less than an average diameter of the immune cell nucleus,
passing a fluid containing the immune cell and the one or more exogenous substances through the plurality of pores to induce a mechanical stress to the immune cell and facilitate introduction of the one or more exogenous substances into the immune cell, and
recirculating the fluid containing the immune cell and the one or more exogenous substances such that the same immune cell passes through the membrane more than once.

2. The method according to claim 1, wherein the average pore diameter of the plurality of pores is no more than an average diameter of the immune cell.

3. The method according to claim 1, wherein the specific flow rate for passing the fluid containing the immune cell and the one or more exogenous substances through the plurality of pores is from 10 nL/s to 1000 nL/s.

4. The method according to claim 1, further comprising, prior to the step of passing the fluid, activating the immune cell from an inactivated to an activated state by exposing the immune cell to one or more activation agents.

5. The method according to claim 4, wherein the method is devoid of introducing one or more conditioning substances into the fluid after the activating step.

6. The method according to claim 1, wherein the immune cell has not been pretreated to alter cell membrane pliability, prior to passing through the membrane.

7. The method according to claim 1, further comprising, prior to passing the immune cell through the membrane, seeding the immune cell into the fluid at a density of no less than 1 × 10⁵ cells/ml and adding the one or more exogenous substances into the fluid.

8. The method according to claim 1, wherein the fluid containing the immune cell and the one or more exogenous substances has a volume of at least 20 mL.

9. The method according to claim 1, wherein the fluid containing the immune cell and the one or more exogenous substances is substantially free of serum when the fluid is passed through the plurality of pores.

10. The method according to claim 10,further comprising providing a time interval of at least 5 minutes to rest the immune cell between any two consecutive passes through the membrane.

11. The method according to claim 1, wherein the immune cell is a T cell.

12. The method according to claim 11, wherein the T cell transfected with the one or more exogenous substances is to be used for immunotherapy.

13. The method according to claim 1, wherein the one or more exogenous substances comprise a nucleic acid configured for expressing one or more exogenous genes for a therapeutic application.

14. The method according to claim 1, wherein, for each pass through the plurality of pores, the number of viable immune cells recovered after passing though the plurality of pores is at least 30% of the number of viable immune cells prior to passing through the plurality of pores.

15. The method according to claim 1, wherein, for each pass through the plurality of pores, the number of immune cells transfected with the one or more exogenous substances is at least 10% of the number of untransfected immune cells prior to passing through the plurality of pores.

16. The method according to claim 1, wherein the number of immune cells transfected with the one or more exogenous substances increases with each additional pass across the membrane.

17. A system for introducing one or more exogenous substances into an immune cell, the system comprising,
a membrane having a plurality of pores configured to allow the immune cell to pass through, wherein the plurality of pores has an average pore diameter which is no less than an average diameter of the immune cell nucleus, and
a pump configured to pass a fluid containing the immune cell and the one or more exogenous substances through the plurality of pores to induce a mechanical stress to the immune cell and facilitate introduction of the one or more exogenous substances into the immune cell,
wherein the pump is further configured to recirculate the fluid containing the immune cell and the one or more exogenous substances such that the same immune cell passes through the membrane more than once.

18. The system according to claim 17, wherein the average pore diameter of the plurality of pores is no more than an average diameter of the immune cell.

19. The system according to claim 17, further comprising a vessel arranged to form an enclosed system such that the membrane is housed within the enclosed system.
